(19) United States
(12) Patent Application Publication
(10) Pub. No.: US 2024/0108055 A1
(43) Pub. Date: Apr. 4, 2024

(54) SMOKE PACKING TOOL

(71) Applicant: Katherine Kilbride, Big Lake, MN (US)

(72) Inventor: Katherine Kilbride, Big Lake, MN (US)

(21) Appl. No.: 18/205,619

(22) Filed: Jun. 5, 2023

Related U.S. Application Data
(60) Provisional application No. 63/411,803, filed on Sep. 30, 2022.

(51) Int. Cl. A24C 5/42; A24F 9/02

(52) U.S. Cl. CPC A24C 5/42; A24F 9/02; A61M 15/06

(57) ABSTRACT

Disclosed herein are apparatuses for packing a cigarette with combustible materials such as tobacco, cannabis, or herbs. The apparatuses include a pair of poking sticks extending from a main body with each of the poking sticks having a different thickness and length from the other to facilitate the packing of the combustible materials within a cigarette. In some embodiments, the sticks can assist the packing of the combustible materials into a pre-rolled cone while helping to maintain proper airflow and an even burn in the pre-rolled cone.

### CROSS-REFERENCE TO RELATED APPLICATIONS

The present application claims the benefit of priority from U.S. Provisional Patent Application No. 63/411,803, filed on Sep. 30, 2022, and entitled “SMOKE PACKING TOOL”, the entire disclosure of which application is hereby incorporated herein by reference.

### TECHNICAL FIELD

The present disclosure relates to apparatuses that can be used as tools to assist smoking devices such as pipes or rolled smoking devices (e.g., cigarettes and cigars).

### BACKGROUND

A cigarette is a narrow cylinder containing a combustible material, typically tobacco, that is rolled into thin paper for smoking. See Wikipedia. The cigarette is ignited at one end, causing it to smolder; the resulting smoke is orally inhaled via the opposite end. Id. Cigarette smoking is the most common method of tobacco consumption. Id. The term cigarette, as commonly used, refers to a tobacco cigarette, but the word is sometimes used to refer to other substances, such as a cannabis cigarette or an herbal cigarette. Id. A cigarette is distinguished from a cigar by its usually smaller size, use of processed leaf, and paper wrapping, which is typically white. Id.

Rolled smoking devices, such as cigarettes, can be hand rolled or rolled by a machine. When rolled manually, it can be difficult to pack the combustible material. This is especially the case when the cigarette is rolled into a cone shape instead of a cylindrical shape. Although tools have been created to assist with the packing process, there is room for improvement.

### SUMMARY

Disclosed herein are apparatuses for packing a cigarette with combustible materials such as tobacco, cannabis, or herbs. The apparatuses include a pair of poking sticks extending from a main body with each of the poking sticks having a different thickness and length from the other to facilitate the packing of the combustible materials within a cigarette. In some embodiments, the sticks can assist the packing of the combustible materials into a pre-rolled cone while helping to maintain proper airflow and an even burn in the pre-rolled cone.

In some examples, an apparatus is configured to effectively pack combustible material into a pre-rolled cone of a pre-rolled cone cigarette. The use of pre-rolled cones, which can be a piece of rolling paper that is wrapped in a conical shape around a cardboard tip, can assist in the hand rolling of a cigarette. However, after filling the pre-rolled cones, the user will often need to pack the inserted combustible materials within the pre-rolled cones. An example purpose of packing the pre-roll cone is to limit gaps or unfilled portions of the cone while maintaining proper airflow to promote an even burn in the pre-roll cone. Ineffectively packing a pre-rolled cone may result in a lopsided pre-roll that may slump and burn inefficiently thereby potentially wasting the combustible materials. With the apparatus configured to effectively pack combustible materials (e.g., tobacco, herbs, cannabis), a pair of poking sticks extend from a main body with each of the poking sticks having a different thickness and length from the other to facilitate the packing of the material effectively to promote, within a pre-rolled cone, proper airflow and an even burn of the packed material.

These and other important aspects of the invention are described more fully in the detailed description below. The invention is not limited to the particular assemblies, apparatuses, methods, and systems disclosed herein. Other embodiments can be used and changes to the described embodiments can be made without departing from the scope of the claims that follow the detailed description.

### BRIEF DESCRIPTION OF THE DRAWINGS

The present disclosure will be understood more fully from the detailed description given below and from the accompanying drawings of various example embodiments of the disclosure.

FIG. 1 illustrates a side perspective view of an example apparatus, in accordance with some embodiments of the present disclosure.

FIG. 2 illustrates a side perspective view of a second example apparatus, in accordance with some embodiments of the present disclosure.

FIG. 3 illustrates a side perspective view of a third apparatus, in accordance with some embodiments of the present disclosure.

### DETAILED DESCRIPTION

The present disclosure will be understood more fully from the detailed description given below and from the accompanying drawings of various example embodiments of the disclosure.

Disclosed herein are apparatuses for packing a cigarette with combustible materials such as tobacco, cannabis, or herbs. The apparatuses include a pair of poking sticks extending from a main body with each of the poking sticks having a different thickness and length from the other to facilitate the packing of the combustible materials within a cigarette. In some embodiments, the sticks can assist the packing of the combustible materials into a pre-rolled cone while helping to maintain proper airflow and an even burn in the pre-rolled cone.

In some examples, an apparatus is configured to effectively pack combustible material into a pre-rolled cone of a pre-rolled cone cigarette. The use of pre-rolled cones, which can be a piece of rolling paper that is wrapped in a conical shape around a cardboard tip, can assist in the hand rolling of a cigarette. However, after filling the pre-rolled cones, the user will often need to pack the inserted combustible materials within the pre-rolled cones. An example purpose of packing the pre-roll cone is to limit gaps or unfilled portions of the cone while maintaining proper airflow to promote an even burn in the pre-roll cone. Ineffectively packing a pre-rolled cone may result in a lopsided pre-roll that may slump and burn inefficiently thereby potentially wasting the combustible materials. With the apparatus configured to effectively pack combustible materials (e.g., tobacco, herbs, cannabis), a pair of poking sticks extend from a main body with each of the poking sticks having a different thickness and length from the other to facilitate the packing of the material effectively to promote, within a pre-rolled cone, proper airflow and an even burn of the packed material.

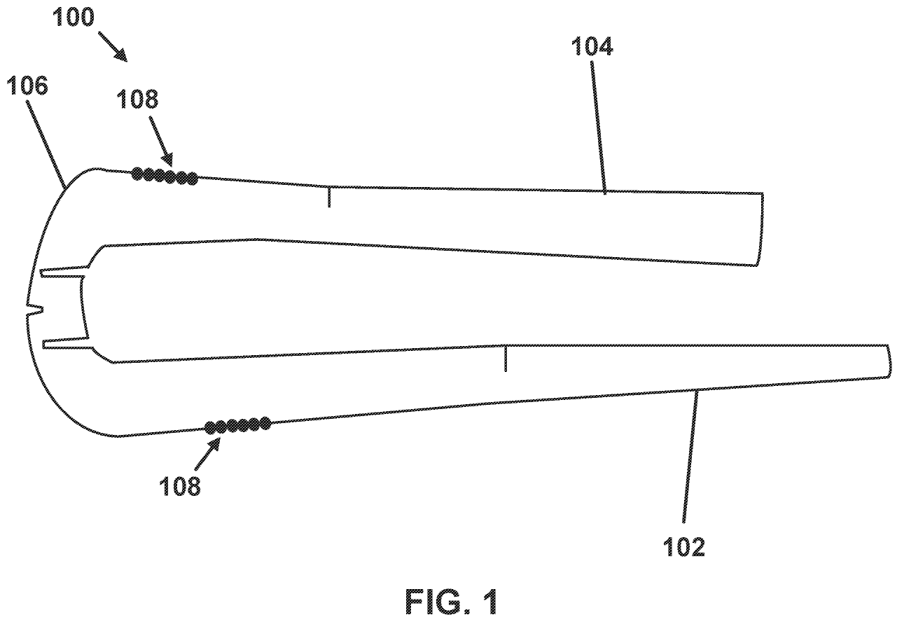

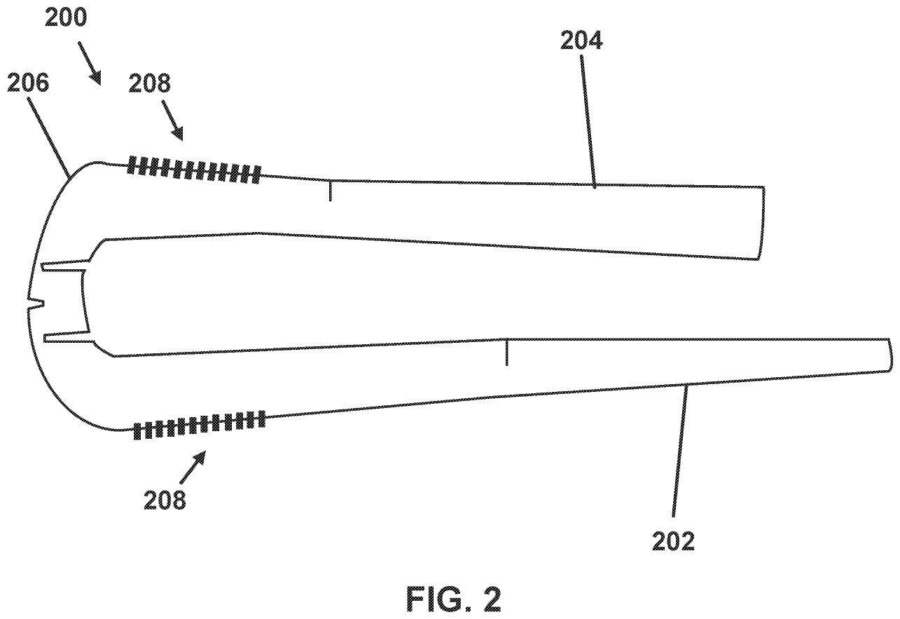

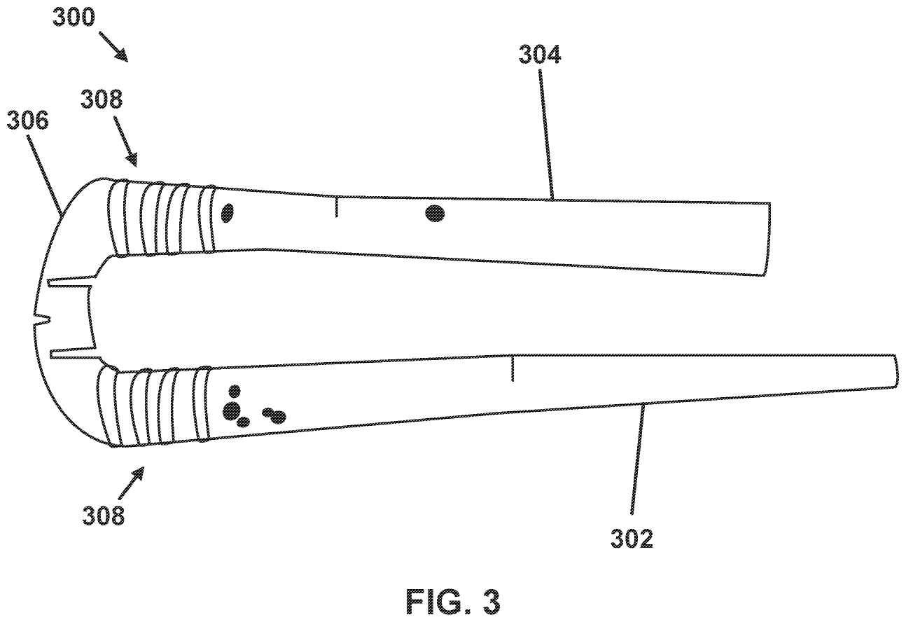

FIGS. 1, 2, and 3 illustrate side perspective views of three different general embodiments of an apparatus, in accordance with some embodiments of the present disclosure.

Referring to the drawings, FIGS. 1, 2, and 3 each show different embodiments of the apparatus (e.g., see apparatuses 100, 200, and 300 depicted in FIGS. 1 to 3, respectively). Each embodiment can be considered a packing tool. Each one of the separate embodiments includes a pair of poking sticks (e.g., see poking sticks 102, 104, 202, 204, 302, and 304 shown in FIGS. 1 to 3, respectively). Each pair of poking sticks includes a first thin poking stick (e.g., see poking sticks 102, 202, and 302) and a second thick poking stick (e.g., see poking sticks 104, 204, and 304). Each pair of the pairs of poking sticks extends from a main body (e.g., see main body 106, 206, or 306 shown in FIGS. 1 to 3 respectively). Each one of the poking sticks, per pair, has a different thickness and length from the other to facilitate the packing of the smoking material within a pre-rolled cone while helping to maintain proper airflow and an even burn in a pre-rolled cone. The apparatus or packing tool can be made from various materials. In the depicted embodiments, the apparatuses are each made from a hemp-based material.

As shown in the drawings, in each of the embodiments, the pair of poking sticks are shown extending from the main body from a distal end of the main body of the packing tool in the same direction. In some other embodiments, the poking sticks extend at various regions of the main body in different directions.

In use, the thin poking stick can be used to pack the combustible material tight for the foundation of the pack. Properly packing the foundation is important since the resulting product will fold at the base and potentially break if the foundation is not initially correctly packed. After the foundation has been properly packed by the thin poking stick, additional combustible material can be added to a pre-rolled cone and the thick poking stick can then be used to pack the combustible material further. In a method of use of the apparatus, the combustible materials are packed less tightly by the thick poking stick compared to the packing of the foundation by the thinner stick, which allows for an effective airflow to move through the packed material.

The variation in the thickness between the two poking sticks in a pair also functions to provide the packing tool with an improved grip. To improve further gripping to the packing tool, the embodiments of FIGS. 1, 2, and 3 also show the pair of poking sticks each having a set of protrusions located on each of the poking sticks proximate to the main body. As shown, the protrusions differ per embodiment (e.g., see protrusions 108, 208, and 308 shown in FIGS. 1 to 3). As shown, each one of the depicted embodiments includes a set of protrusions and, in the set, the protrusions adjacent to the first stick are similar to the protrusions of the second stick. However, in other embodiments, the set can have different types of protrusions on the first and second sticks. With each embodiment, the set of protrusions functions as finger grips for enhancing the user's gripping of the apparatus or packing tool. As shown in the embodiments of FIGS. 1, 2, and 3, the set of protrusions can vary in shape, size, and amounts of protrusions in the set per stick.

The differences in the lengths of the poking sticks with the thick poking stick being shorter than the thinner poking stick provides functional limits so that the thick poking stick cannot go as deep into the pre-rolled cone as the thin poking stick thereby limiting the thick poking stick's access to the foundation of the smoking material.

In some embodiments, the apparatus can be smooth without grips. In some embodiments, the two poking sticks can be smooth without grips. In some embodiments, one of the two poking sticks can be smooth without grips.

It is to be understood that although the drawings show the packing tool or the apparatus having two poking sticks, alternative embodiments can include a packing tool having more than two poking sticks with the sticks each having different functional characteristics by each having different lengths and thicknesses.

In the foregoing specification, embodiments of the disclosure have been described with reference to specific example embodiments thereof. It will be evident that various modifications can be made thereto without departing from the broader spirit and scope of embodiments of the disclosure as set forth in the following claims. The specification and drawings are, accordingly, to be regarded in an illustrative sense rather than a restrictive sense.

What is claimed is:

1. An apparatus, comprising:
a main body; and
a pair of poking sticks, the pair including a first stick that is thinner and longer than a second stick of the pair.

2. The apparatus of claim 1, wherein the respective widths of cross-sections of the first stick become smaller towards the end of the first stick opposing the main body.

3. The apparatus of claim 2, wherein the respective widths of cross-sections of the second stick become smaller towards the end of the second stick opposing the main body.

4. The apparatus of claim 2, wherein the respective widths of cross-sections of the second stick first become smaller towards the end of the second stick opposing the main body and then become larger afterward until the end of the second stick.

5. The apparatus of claim 1, wherein cross-sections of the first stick are circular.

6. The apparatus of claim 5, wherein the cross-sections of the second stick are circular.

7. The apparatus of claim 1, wherein a first interface between the main body and the first stick includes first circular protrusions configured to be gripped.

8. The apparatus of claim 7, wherein a second interface between the main body and the second stick includes second circular protrusions configured to be gripped.

9. The apparatus of claim 1, wherein a first interface between the main body and the first stick includes first rectangular protrusions configured to be gripped.

10. The apparatus of claim 9, wherein a second interface between the main body and the second stick includes second rectangular protrusions configured to be gripped.

11. The apparatus of claim 1, wherein a first interface between the main body and the first stick includes first spiral protrusions configured to be gripped.

12. The apparatus of claim 11, wherein a second interface between the main body and the first stick includes second spiral protrusions configured to be gripped.

13. The apparatus of claim 1, wherein the main body is u-shaped.

14. An apparatus, comprising:
a main body; and
a pair of poking sticks, the pair including a first stick that is thinner and longer than a second stick of the pair, wherein the respective widths of cross-sections of the first stick become smaller towards the end of the first stick opposing the main body, and wherein a first interface between the main body and the first stick includes protrusions configured to be gripped.

15. The apparatus of claim 14, wherein the protrusions include circular protrusions configured to be gripped.

16. The apparatus of claim 14, wherein the protrusions include rectangular protrusions configured to be gripped.

17. The apparatus of claim 14, wherein the protrusions include spiral protrusions configured to be gripped.

18. An apparatus, comprising:
a main body; and
a pair of poking sticks, the pair including a first stick that is thinner and longer than a second stick of the pair,
wherein the respective widths of cross-sections of the first stick become smaller towards the end of the first stick opposing the main body,
wherein widths of cross-sections of the second stick first become smaller towards the end of the second stick opposing the main body and then become larger afterward until the end of the second stick,
and wherein a first interface between the main body and the first stick includes protrusions configured to be gripped.

19. The apparatus of claim 18, wherein the protrusions include spiral protrusions configured to be gripped.

20. The apparatus of claim 18, wherein the protrusions include rectangular protrusions or circular protrusions configured to be gripped.
